(19) United States
(12) Patent Application Publication
(10) Pub. No.: US 2026/0200779 A1
(43) Pub. Date: Jul. 16, 2026

(54) Bioengineered Moss for Arsenic Removal from Water

(71) Applicant: Boss Moss LLC, Spokane, WA (US)

(72) Inventors: Lance Dunn, Spokane, WA (US); Darren Thomas Melville, Spokane, WA (US); Ty Mcconnell Patterson, Newberg, OR (US)

(21) Appl. No.: 19/396,313

(22) Filed: Nov. 20, 2025

Related U.S. Application Data
(60) Provisional application No. 63/724,210, filed on Nov. 22, 2024.

(51) Int. Cl. C02F 3/32; C02F 101/10; C02F 103/00; C02F 103/06; C12N 9/10; C12N 9/22; C12N 15/113; C12N 15/82

(52) U.S. Cl. CPC C02F 3/327; C12N 9/1007; C12N 9/226; C12N 15/113; C12N 15/8243; C12Y 201/01137; C02F 2101/103; C02F 2103/007; C02F 2103/06; C12N 2310/20

(57) ABSTRACT

The composition of a genetically engineered moss used for water purification is disclosed. The genetically engineered moss comprises an arsenic methyltransferase gene. In use, the genetically engineered moss contacts arsenic-contaminated water and absorbs arsenic from the water, then methylates the absorbed arsenic within the moss to produce a non-toxic trimethylarsenic compound. A system for water purification is also provided, comprising a container for holding contaminated water and a genetically engineered moss disposed within the container. The moss comprises an arsenic methyltransferase gene configured to methylate absorbed arsenic to produce a non-toxic trimethylarsenic compound.

### CROSS-REFERENCE TO RELATED APPLICATIONS

This application claims priority to U.S. Provisional Patent Application No. 63/724,210, filed Nov. 22, 2024, entitled Bioengineered Moss for Arsenic Removal from Water, which is herein incorporated by reference.

### FIELD OF THE INVENTION

The present invention generally relates to water purification systems, and more particularly to bioengineered moss-based filtration system for removing arsenic and other heavy metals from contaminated water sources.

### BACKGROUND

Water contamination is a global issue affecting millions of people worldwide. Among the various contaminants found in water sources, arsenic poses a significant threat to human health. Arsenic is a naturally occurring element that can enter water supplies through the dissolution of minerals and ores. It is also introduced into the environment through industrial processes, agricultural runoff, and mining activities. It is estimated among 170 million people globally drink water with dangerous arsenic levels, 70 countries that are heavily affected by arsenic-contaminated water, and around 24,000 deaths are caused by arsenic poisoning each year.

Furthermore, the presence of arsenic in drinking water has been linked to numerous health problems, including various types of cancer, cardiovascular diseases, and developmental disorders. The World Health Organization (WHO) has established a guideline value of 10 micrograms per liter as the maximum concentration of arsenic in drinking water. However, many water sources in affected areas contain arsenic levels far exceeding this limit.

Conventional methods for removing arsenic from water include reverse osmosis, ion exchange, and adsorption using activated alumina or iron-based adsorbents. While these methods can be effective, they often require complex infrastructure, high operational costs, and regular maintenance. Additionally, some of these techniques generate hazardous waste products that require proper disposal, creating additional environmental concerns.

As such, there is thus a need for addressing these and/or other issues associated with the prior art.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, the composition of a genetically engineered moss used for water purification is provided. The genetically engineered moss comprises an arsenic methyltransferase gene. In use, the genetically engineered moss contacts arsenic-contaminated water and absorbs arsenic from the water, then methylates the absorbed arsenic within the moss to produce a non-toxic trimethylarsenic compound.

According to other aspects of the present disclosure, the genetically engineered moss may be capable of absorbing up to 9 milligrams of arsenic per gram of moss. The moss may be capable of removing up to 82% of arsenic from contaminated water within one hour. The moss may be a bryophyte, allowing for arsenic uptake through its entire surface area when in contact with water. The arsenic methyltransferase gene may be derived from the plant Chlamydomonas reinhardtii. The genetic modification of the moss may be achieved using CRISPR technology.

According to another aspect of the present disclosure, a system for water purification is provided, comprising a container for holding contaminated water and a genetically engineered moss disposed within the container. The moss comprises an arsenic methyltransferase gene configured to methylate absorbed inorganic arsenic to produce a non-toxic trimethylarsenic compound.

According to other aspects of the present disclosure, the system may include one or more of the following features. The system may include a cylindrical container possible of being incorporated into a water bottle for portable, everyday use. The system may involve periodically replacing the moss in the container. The used moss may be repurposed as a compostable fertilizer due to the non-toxic nature of the trimethylarsenic. The system may include a container configured to treat well water in areas affected by arsenic contamination.

According to another aspect of the present disclosure, the system may involve a container configured to treat stream water in areas affected by arsenic contamination. The system may also involve a container configured to treat contaminated water in wetland areas.

According to other aspects of the present disclosure, the water purification system may be scalable for use in various applications, from residential systems to large industrial plants. The system may be designed for easy installation and maintenance in existing water treatment infrastructure.

The foregoing general description of the illustrative embodiments and the following detailed description thereof are merely exemplary aspects of the teachings of this disclosure and are not restrictive.

### BRIEF DESCRIPTION OF THE DRAWINGS

Non-limiting and non-exhaustive examples are described with reference to the following figures.

FIG. 1 illustrates a water filtration system utilizing genetically modified moss for water purification, according to aspects present in the disclosure.

FIG. 2 illustrates a water purification system for removing heavy metals from water, according to an embodiment.

FIG. 3 illustrates a water purification system comprising multiple components for processing and purifying water, according to aspects of the present disclosure.

FIG. 4 illustrates an orthogonal view of a water purification system, according to an embodiment.

FIG. 5 illustrates a water purification system for removing arsenic from contaminated water, according to aspects of the present disclosure.

FIG. 6 illustrates a biomethylation process for arsenic compounds, according to an embodiment.

FIG. 7 illustrates a flowchart of a method for purifying contaminated water using genetically engineered moss, according to aspects of the present disclosure.

### DETAILED DESCRIPTION

In recent years, there has been growing interest in developing more sustainable and cost-effective approaches to water purification. Bioremediation, which utilizes living organisms to remove or neutralize contaminants, has emerged as a promising field of study. Various microorganisms and plants have been investigated for their ability to accumulate or transform arsenic into less toxic forms.

Moss, a type of bryophyte, has garnered attention in environmental research due to its unique properties. Mosses are known for their ability to absorb and retain water, as well as their capacity to accumulate various elements from their surroundings. These characteristics make moss a potential candidate for water purification applications.

Advancements in genetic engineering and synthetic biology have opened up new possibilities for enhancing the natural capabilities of organisms. By modifying the genetic makeup of plants or microorganisms, it may be possible to improve their ability to remove specific contaminants from water or to introduce new functionalities that aid in the purification process.

As the demand for clean water continues to grow, there is an ongoing need for innovative, efficient, and environmentally friendly water treatment solutions. Developing technologies that can effectively remove arsenic and other heavy metals from water sources, while being sustainable and adaptable to various settings, remains a critical challenge in the field of water purification.

As disclosed herein, the moss filtration system effectively overcomes the deficiencies in current systems. Additionally, the modular, lower-priced, eco-friendly, and simple method revolutionizes a new approach to a common global problem that has plagued many nations.

### Definitions and Use of Figures

Some of the terms used in this description are defined below for easy reference. The presented terms and their respective definitions are not rigidly restricted to these definitions—a term may be further defined by the term's use within this disclosure. The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application and the appended claims, the term “or” is intended to mean an inclusive “or” rather than an exclusive “or”. That is, unless specified otherwise, or is clear from the context, “X employs A or B” is intended to mean any of the natural inclusive permutations. That is, if X employs A, X employs B, or X employs both A and B, then “X employs A or B” is satisfied under any of the foregoing instances. As used herein, at least one of A or B means at least one of A, or at least one of B, or at least one of both A and B. In other words, this phrase is disjunctive. The articles “a” and “an” as used in this application and the appended claims should generally be construed to mean “one or more” unless specified otherwise or is clear from the context to be directed to a singular form.

Various embodiments are described herein with reference to the figures. It should be noted that the figures are not necessarily drawn to scale, and that elements of similar structures or functions are sometimes represented by like reference characters throughout the figures. It should also be noted that the figures are only intended to facilitate the description of the disclosed embodiments—they are not representative of an exhaustive treatment of all possible embodiments, and they are not intended to impute any limitation as to the scope of the claims. In addition, an illustrated embodiment need not portray all aspects or advantages of usage in any particular environment.

An aspect or an advantage described in conjunction with a particular embodiment is not necessarily limited to that embodiment and can be practiced in any other embodiments even if not so illustrated. References throughout this specification to “some embodiments” or “other embodiments” refer to a particular feature, structure, material or characteristic described in connection with the embodiments as being included in at least one embodiment. Thus, the appearance of the phrases “in some embodiments” or “in other embodiments” in various places throughout this specification are not necessarily referring to the same embodiment or embodiments. The disclosed embodiments are not intended to be limiting of the claims.

### Descriptions of Exemplary Embodiments

The following description sets forth exemplary embodiments of the present disclosure. It should be recognized, however, that the description is provided for illustrative purposes only and is not intended to limit the scope of the claimed invention. Rather —-variations, modifications, and alternative configurations will be apparent to those skilled in the art and are considered to fall within the scope of the present disclosure.

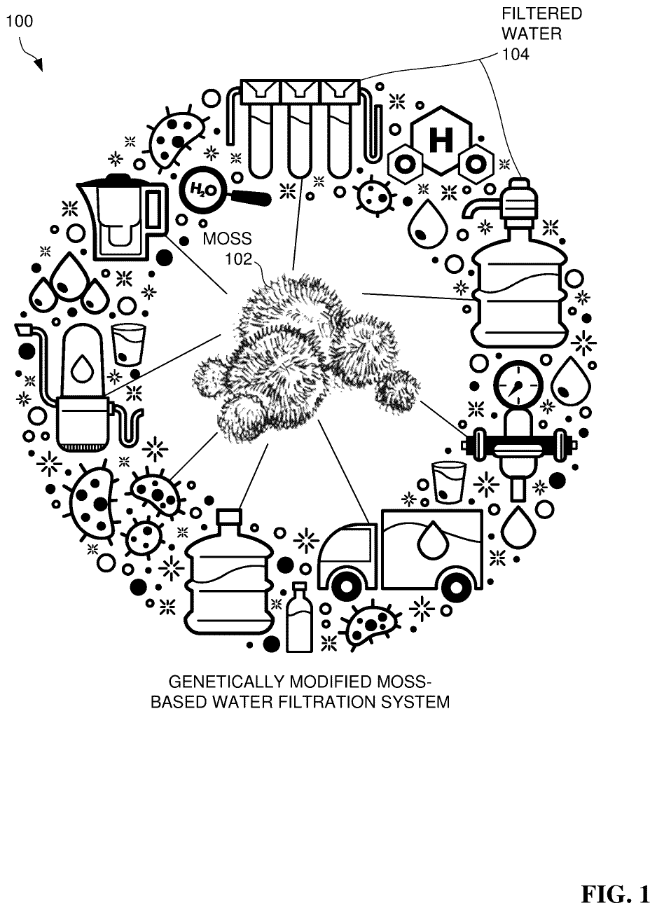

FIG. 1 illustrates a water filtration system 100 utilizing genetically modified moss 102 for water purification. In the depicted embodiment, system 100 comprises a plurality of components arranged in a circular layout to represent various stages and elements of the filtration process. The moss 102 is centrally located and functions as the primary filtration medium. In one embodiment, moss 102 may be depicted as a cluster of small, fuzzy structures to visually represent its biological nature and high surface area for contaminant absorption.

Surrounding the moss 102 are various icons and symbols representative of the water purification process. These may include, for example, water droplets, molecular structures, and containers, which together illustrate the progression of water from a contaminated state to a filtered state 104. The system 100 also includes representations of laboratory equipment, such as test tubes and beakers, suggesting the scientific processes involved in developing and monitoring the genetically modified moss-based filtration.

The system 100 may depict a variety of water containers, ranging in size from small glasses to large jugs, indicating potential applications of the technology at multiple scales. In some embodiments, the system may be employed for personal use, while in others it may be configured for community-level water purification. The filtered water 104 may be represented by a water droplet icon connected to the outer circle, symbolizing the final output of the purification process.

The circular arrangement of these elements emphasizes the cyclical and sustainable nature of the filtration system, with the moss 102 at its core continuously processing and purifying water to produce the filtered water 104.

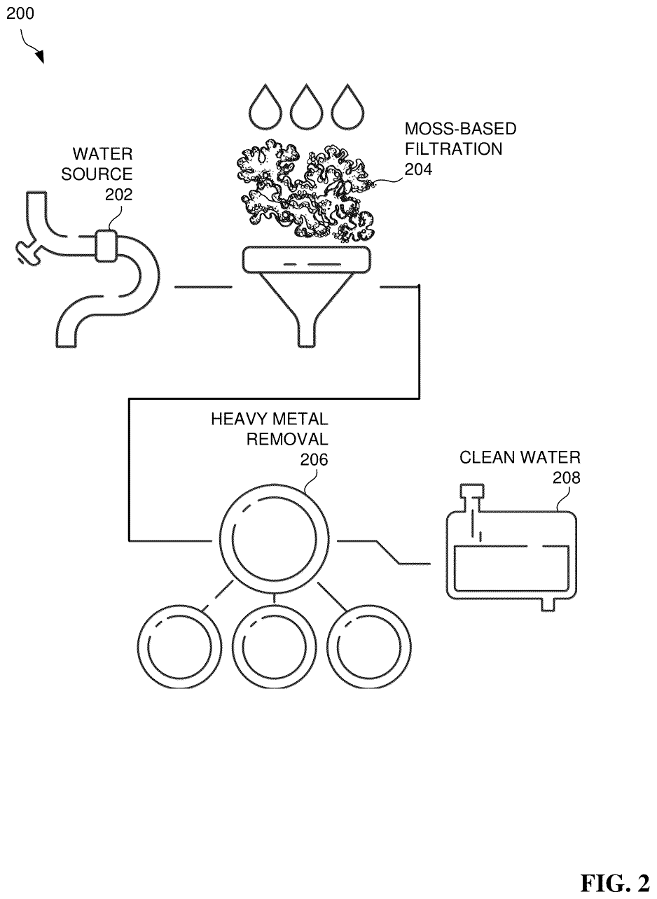

FIG. 2 illustrates an alternative embodiment of a water purification system 200 configured for removing heavy metals from contaminated water. The system 200 comprises a water source 202, a moss-based filtration unit 204, a heavy metal removal unit 206, and a clean water container 208.

The water source 202 may be a faucet, tap, or other supply input from which contaminated water is directed into the system. The water flows into moss-based filtration unit 204, which includes a funnel-shaped housing containing moss material. The moss-based filtration unit 204 may serve as a primary filtration stage, where genetically engineered moss begins the process of heavy metal removal.

After initial treatment by the moss-based filtration unit 204, the water flows into a heavy metal removal unit 206. This unit is represented as a series of circular chambers or stages, each of which may implement further filtration techniques such as chemical treatment, or other purification methods that complement the moss-based filtration.

Finally, the purified water may then enter the clean water container 208, which serves as the final collection point in the system. The container 208 may be depicted as a tank, bottle, or other vessel suitable for safely storing the treated water until use. This container represents the end point of the purification process, where the water, now free of heavy metals, is stored for use.

In the illustrated embodiment, system 200 is configured for sequential operation, where water flows from water source 202 through moss-based filtration unit 204, then through heavy metal removal unit 206, and finally into clean water container 208. Directional arrows in FIG. 2 may be used to indicate the progression of water through the system, illustrating the step-by-step purification process.

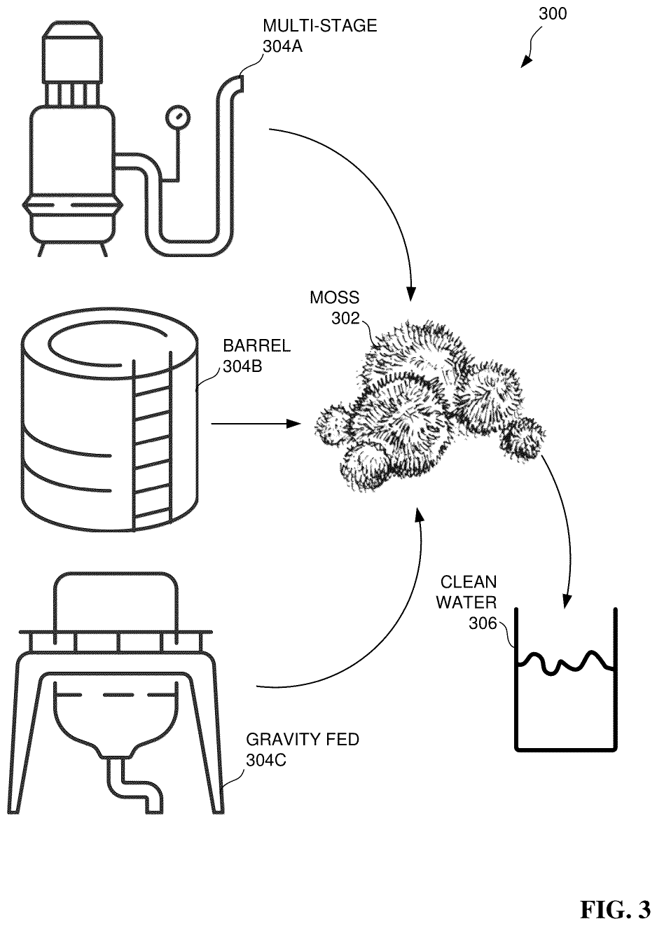

FIG. 3 illustrates another embodiment of a water purification system 300 comprising a moss-based treatment component 302 and multiple apparatus configurations for processing and purifying water. The system 300 may be configured to interface with a multi-stage apparatus 304A, a barrel system 304B, and a gravity-fed apparatus 304C. These apparatuses may vary in complexity and application scale, ranging from household units to industrial or off-grid systems. The moss 302 functions as a common purification element across these configurations.

The multi-stage apparatus 304A, shown at the top of FIG. 3, may be a pressurized system including multiple sequential treatment chambers. In certain embodiments, this configuration enables enhanced purification efficiency and may be particularly well-suited for high-volume or industrial applications. The barrel 304B, shown in the middle of the figure, may take the form of a cylindrical storage or treatment vessel that may be used for storing or treating water. This simpler design could be appropriate for residential or small-scale use. The gravity-fed apparatus 304C, shown at the bottom of the figure, utilizes gravity to move water through the filtration stages. This configuration offers an energy-efficient option for water treatment.

The moss 302 is centrally located in the diagram and may be operatively connected to each of the three apparatus configurations. In one embodiment, directional arrows indicate fluid communication between moss 302 and the respective systems 304A, 304B, and 304C. This arrangement illustrates the adaptability of moss 302 as a modular and versatile filtration medium capable of integration with a variety of water treatment scenarios.

A water container 306, labeled as “CLEAN WATER,” is depicted on the right side of the diagram. An arrow connects the moss 302 to this container 306, indicating that the purified water is collected or stored here after treatment. This arrangement suggests a versatile system capable of handling water from different sources or through different treatment methods, all utilizing the moss 302 as a common purification element.

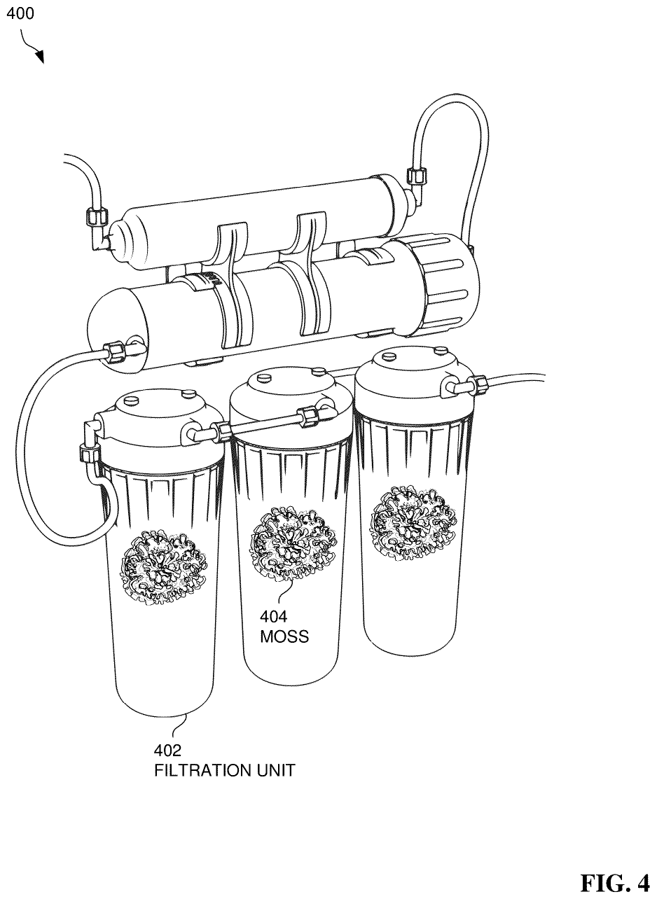

FIG. 4 illustrates an orthogonal view of a water purification system 400. In this embodiment, system 400 comprises a filtration unit 402 and moss 404. The filtration unit 402 includes three vertically arranged cylindrical chambers, each containing moss 404. The chambers may be constructed from transparent material to visually demonstrate the interaction between the moss and the water flowing through it. The moss 404 appears as a clump-like structure within each chamber, providing a large surface area for water purification.

The filtration unit 402 is connected to a series of pipes and connectors located at the top of the system. These connectors may include two horizontal cylindrical components joined to the vertical chambers. In one embodiment, the upper horizontal component appears larger in diameter than the lower component. Flexible tubing extends from both ends of the upper cylindrical component to provide inlet and outlet pathways for water. This configuration allows for controlled water flow into and out of the filtration chambers.

The three vertical chambers of the filtration unit 402 may be secured together using bands, clamps, or other fastening mechanisms. Each chamber may be equipped with a removable top cap to allow for periodic maintenance or replacement of moss 404. In some embodiments, the bottom of each chamber narrows, potentially to promote directed water flow and facilitate drainage, thereby improving filtration efficiency. This design ensures efficient water movement through the moss 404 and easy maintenance of the system.

During operation of the water purification system 400 water flows sequentially through each of the vertically stacked chambers containing moss 404. As water passes through the moss-filled chambers, the moss 404 interacts with the water, presumably to remove contaminants or purify the water in some manner. The multi-stage design of the filtration unit 402 may improve the overall purification performance by exposing the water to repeated cycles of moss-based filtration, thereby potentially reducing the concentration of contaminants with each stage of the purification process.

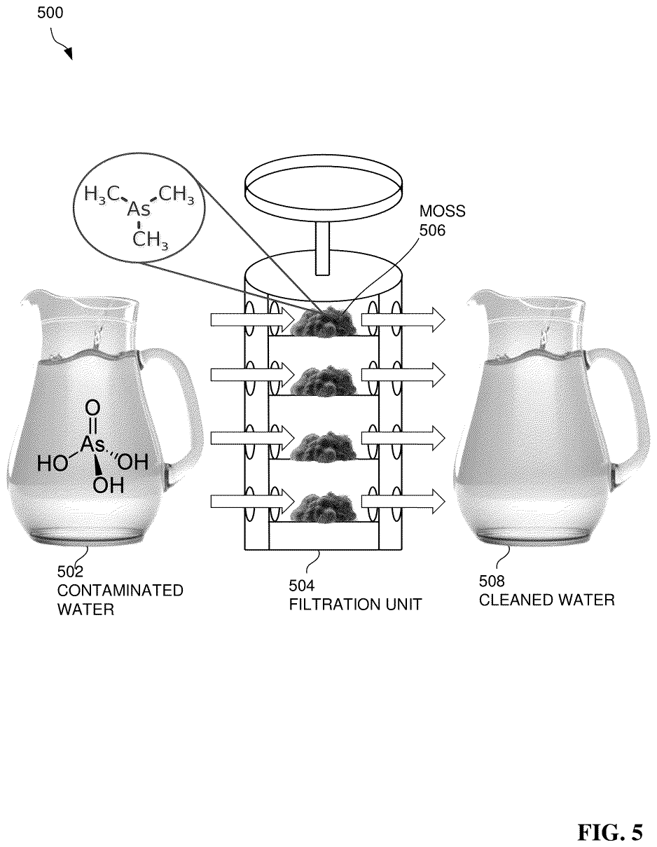

FIG. 5 illustrates a water purification system 500 specifically configured for the removal of arsenic from contaminated water. The system 500 comprises a contaminated water container 502, a filtration unit 504, a moss medium 506, and a clean water container 508.

The contaminated water container 502 stores the arsenic-laden water prior to treatment. This is represented by a chemical structure shown in the figure. This container serves as the starting point for the purification process, holding the water that requires treatment.

The filtration unit 504 is positioned between the contaminated water container 502 and the cleaned water container 508. The filtration unit 504 includes multiple layers or stages, with each containing moss 506. This layered approach allows for thorough interaction between the contaminated water and the purifying moss.

The moss 506 is arranged in layers within the filtration unit 504 to promote thorough contact with the contaminated water. As water flows through these layers, the moss 506 absorbs and processes arsenic from the solution. In certain embodiments, the moss 506 may facilitate a chemical transformation of inorganic arsenic into a less toxic organic form. For example, FIG. 5 includes a chemical structure depicting trimethylarsenic, representing the transformation process. This biomethylation capability underscores the moss's dual role as both a physical filtration medium and a biochemical detoxification agent.

Once treated, the water exits the filtration unit 504 and enters the cleaned water container 508. The cleaned water container 508 stores the purified water now with reduced arsenic content. In preferred embodiments, the arsenic concentration is reduced to levels below applicable drinking water standards. The system 500 demonstrates an integrated purification approach in which water contaminated with arsenic is transformed into clean water by using moss-based filtration unit 504.

With continued reference to FIG. 5, the pitcher of water on the left may represent undrinkable well water containing elevated concentrations of inorganic arsenic or other heavy metals. Arsenic may be continuously being deposited into these wells through their water accumulation systems, weathering bedrock and dissolving arsenic from the soil, as our device is placed into a well, and moss filtration system may come into contact with this contaminated water. The moss may absorb the arsenic through phosphate transporter channels, storing it within its tissues (e.g. up to nine milligrams of arsenic per gram of boss moss). Unlike most hyper accumulators of arsenic, which uptake through their roots, storing it in their tissue, the moss filtration system disclosed herein is a bryophyte, meaning it's able to uptake the arsenic anywhere it comes into contact with water.

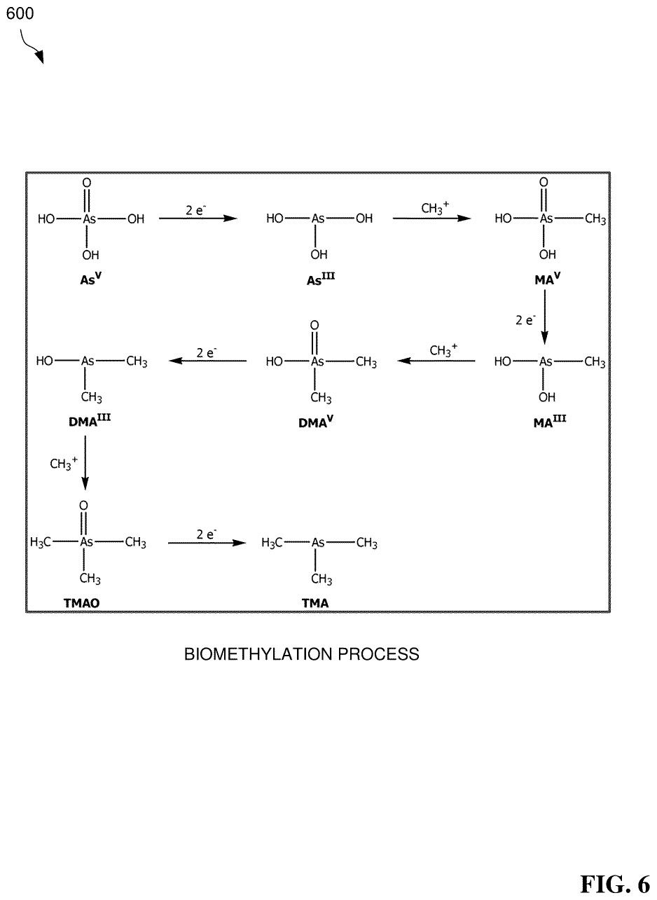

As a result of this massive usable surface area, the moss filtration system dislosed herein may be able to remove up to 82% of arsenic within one hour (which for context brings water over five times the drinkable limit into a drinkable range during that hour). This is represented in by the pitcher of water on the right in FIG. 6, showing clean, drinkable water.

FIG. 6 illustrates the biomethylation process diagram 600 for arsenic compounds. This diagram details the step-by-step process by which inorganic arsenic is transformed into less toxic organic forms through a series of methylation and reduction reactions.

The process begins with AsV, which is inorganic arsenic in its pentavalent form. AsV undergoes a reduction reaction, gaining two electrons (2e−) to form AsIII, the trivalent form of inorganic arsenic. This initial reduction step is crucial for the subsequent methylation reactions.

AsIII then undergoes methylation by gaining a methyl group (CH3+) to form MAV, monomethylarsonic acid. This is the first step in the organic transformation of the arsenic compound. MAV is then reduced by gaining two electrons to form MAIII, monomethylarsonous acid.

MAIII undergoes a second methylation step, gaining another methyl group to form DMAV, dimethylarsinic acid. This compound is then reduced by gaining two electrons to form DMAIII, dimethylarsinous acid.

DMAIII undergoes a final methylation step, gaining a third methyl group to form TMAO, trimethylarsine oxide. Finally, TMAO is reduced by gaining two electrons to form TMA, trimethylarsine, which is the end product of the biomethylation process.

Each step in the process is clearly depicted with the chemical structures of the arsenic compounds and the arrows indicating the direction of the reactions. The process demonstrates the progressive methylation and reduction of arsenic compounds, ultimately converting the toxic inorganic arsenic into a less toxic organic form. This biomethylation process is key to understanding how the genetically engineered moss in the water purification system detoxifies arsenic.

With continued reference to FIG. 6, the moss filtration system may perform a process called biomethylation. Biomethylation may turn toxic inorganic arsenic into trimethylarsenic, a completely non toxic molecule, allowing the moss filtration system to be utilized as a compostable fertilizer. In one embodiment, this may be accomplished with the help of the arsenic methyltransferase gene from the plant chlamydomonas reinhardtii. To utilize this gene, CRISPR may be used, entailing the creation of a template RNA strand that encodes for the arsenic methyltransferase gene, attaching it to a cas9 which will then precisely cut the DNA, utilizing the cells of the moss natural DNA repair process to modify the DNA to code for this inserted gene.

In some embodiments, the genetic modification of moss may be accomplished through homologous recombination. Certain moss species, such as Physcomitrium patens, exhibit an unusually high efficiency of homologous recombination compared to most higher plants. By introducing a DNA construct flanked by regions of sequence hology to a genomic locus of interest, it is possible to achieve targeted integration or replacement of sequences using the cell's natural DNA repair pathways. This approach permits precise and stable modification of the moss genome without requiring nuclease-mediated double-strand breaks.

In other embodiments, genetic modification may be achieved through protoplast transformation. Moss protoplasts may be prepared by enzymatically removing the cell wall, thereby generating intact cells with exposed membranes. Exogenous nucleic acids may then be introduced into the protoplasts through chemical treatment (e.g., polyethylene glycol) or physical methods (e.g., electroporation), which transiently increase membrane permeability. Once internalized, the introduced DNA may undergo random integration or hologous recombination, depending on the construct design. Protoplast transformation provides a flexible and scalable means of delivering nucleic acids into moss cells.

In still other embodiments, Agrobacterium tumefaciens or related species may be employed to mediate DNA transfer into moss cells. During infection, Agrobacterium naturally transfers a segment of DNA (T-DNA) from its plasmid into the genome of plant hosts. By engineering this T-DNA region to contain a desired sequence the bacterium can serve as a biological vector for moss transformation. Co-cultivation of moss tissues or cell suspensions with engineered Agrobacterium strains may result in stable integration of the heterologous sequence into the moss genome. This method provides a biologically mediated alternative to chemical or physical transformation techniques.

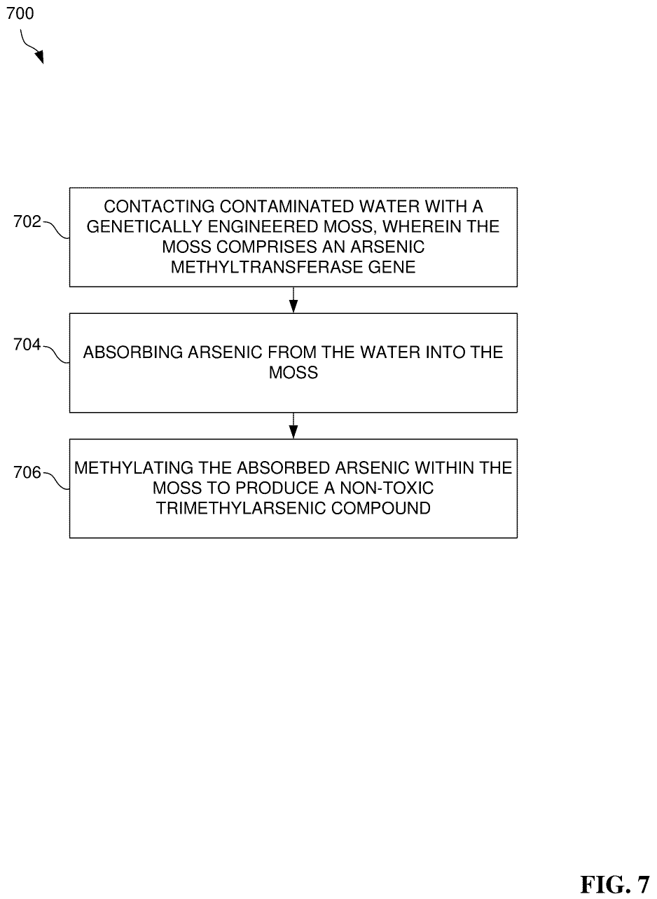

FIG. 7 illustrates a flowchart of method 700 for purifying contaminated water using genetically engineered moss. The method 700 comprises three primary steps: contacting, absorption, and methylation. While these steps are presented in a particular order, it should be understood that variations in sequence, integration, or repetition may be employed in certain implementations.

The method begins with step 702, which involves contacting contaminated water with a genetically engineered moss. In one embodiment, the moss comprises an inserted arsenic methyltransferase gene, which is essential for the arsenic detoxification process. This step represents the initial interaction between the contaminated water and the engineered moss.

Following step 702, the method 700 proceeds to step 704. In this step, arsenic is absorbed from the water into the moss. This absorption process is facilitated by the unique properties of the genetically engineered moss, which has been designed to efficiently uptake arsenic from the water.

The final step of the method 700 is step 706. This step involves methylating the absorbed arsenic within the moss to produce a non-toxic trimethylarsenic compound. This methylation process, enabled by the arsenic methyltransferase gene introduced into the moss, is the key to transforming the toxic inorganic arsenic into a less harmful organic form.

The flowchart depicts the sequential nature of these steps, with each step leading directly to the next. The method 700 outlines a process that combines genetic engineering, absorption, and chemical transformation to address water contamination. This approach utilizes the specific genetic modification of the moss to enable both the uptake and detoxification of arsenic from contaminated water sources, providing an efficient and environmentally friendly solution to arsenic contamination in water.

A number of implementations have been described. Nevertheless, it will be understood that various modifications may be made without departing from the spirit and scope of the disclosure. Accordingly, other implementations are within the scope of the following claims.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

The embodiments described herein included the one or more modes known to the inventor for carrying out the claimed subject matter. Of course, variations of those embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

What is claimed is:

1. A composition for water purification, comprising:
a genetically engineered moss comprising an arsenic methyltransferase gene, wherein the moss is configured to absorb arsenic from contaminated water and methylate the absorbed arsenic to produce a non-toxic trimethylarsenic compound.

2. The composition of claim 1, wherein the genetically engineered moss is capable of absorbing up to 9 milligrams of arsenic per gram of moss.

3. The composition of claim 1, wherein the genetically engineered moss is capable of removing up to 82% of arsenic from contaminated water within one hour.

4. The composition of claim 1, wherein the genetically engineered moss is a bryophyte.

5. The composition of claim 4, wherein the bryophyte is capable of arsenic uptake through its entire surface area when in contact with water.

6. The composition of claim 1, wherein the arsenic methyltransferase gene is derived from the algae chlamydomonas reinhardtii.

7. The composition of claim 1, wherein the genetic modification of the moss is achieved using CRISPR-Cas9 technology.

8. The composition of claim 1, further comprising a filtration unit containing the genetically engineered moss.

9. The composition of claim 8, wherein the filtration unit comprises multiple layers of the genetically engineered moss.

10. The composition of claim 9, wherein the filtration unit is configured for use in a gravity-fed water purification system.

11. The composition of claim 9, wherein the filtration unit is configured to treat well water in areas affected by arsenic contamination.

12. The composition in claim 9, wherein the filtration unit is configured for wetland use.

13. The composition of claim 9, where the filtration unit is configured for stream water use.

14. The composition of claim 10, wherein the filtration unit is portable.

15. The composition of claim 10, wherein the filtration unit is in the shape of a cylinder.

16. The composition of claim 15, wherein the filtration unit is configured for use in a water bottle.

17. A system for water purification, comprising:
a container configured to hold contaminated water;
a filtration unit disposed within the container, the filtration unit comprising a genetically engineered moss, wherein the genetically engineered moss comprises an arsenic methyltransferase gene and is configured to:
absorb arsernic from the contaminated water;
methylate the absorbed arsenic to produce a non-toxic trimethylarsenic compound; and
an outlet configured to allow purified water to exit the container after treatment by the genetically engineered moss.

18. The system of claim 17, wherein the genetically engineered moss is a bryophyte.

19. The system of claim 17, the filtration unit is configured to treat well water in areas affected by arsenic contamination.

20. The system of claim 17, wherein the filtration unit is configured for wetland use.
